(19) United States
(12) Patent Application Publication
(10) Pub. No.: US 2025/0311994 A1
(43) Pub. Date: Oct. 9, 2025

(54) X-RAY CT DEVICE, MEDICAL IMAGE PROCESSING DEVICE, AND MEDICAL IMAGE PROCESSING METHOD

(71) Applicant: CANON MEDICAL SYSTEMS CORPORATION, Tochigi (JP)

(72) Inventors: Yudai YAMAZAKI, Nasushiobara (JP); Kosuke ARITA, Kawasaki (JP); Asateru KIMURA, Yokohama (JP); Yohei MURAGUCHI, Nasushiobara (JP)

(73) Assignee: CANON MEDICAL SYSTEMS CORPORATION, Tochigi (JP)

(21) Appl. No.: 19/098,438

(22) Filed: Apr. 2, 2025

(30) Foreign Application Priority Data
Apr. 3, 2024 (JP) 2024-060412

(51) Int. Cl. A61B 6/00; A61B 6/03; A61B 6/06; A61B 6/42; G01T 1/24; G16H 30/40

(52) U.S. Cl. CPC A61B 6/5258; A61B 6/032; G01T 1/247; G16H 30/40; A61B 6/06; A61B 6/4241

(57) ABSTRACT

An X-ray CT device according to an embodiment includes a memory, and processing circuitry. The processing circuitry: estimates a component amount of an in vivo component and component amounts of a noise component and an unknown component based on a medical image in which a living body is depicted by using a physical model that takes a noise component and an unknown component into account; calculates likelihood of the component amounts with respect to the physical model as a first evaluation value; calculates a second evaluation value based on morphological features of the in vivo component, the noise component, and the unknown component; and updates the component amount of the in vivo component as well as the component amounts of the noise component and the unknown component based on the first evaluation value and the second evaluation value.

### CROSS-REFERENCE TO RELATED APPLICATIONS

This application is based upon and claims the benefit of priority from Japanese Patent Application No. 2024-060412, filed on Apr. 3, 2024; the entire contents of which are incorporated herein by reference.

### FIELD

Embodiments described herein relate generally to an X-ray CT device, a medical image processing device, and a medical image processing method.

### BACKGROUND

Image information in diagnosis by physicians is expected to play an important role. In X-ray computed tomography (CT), the internal structure of a target is imaged based on the transmission amount of X-rays. Furthermore, in an optical image, a target is imaged based on reflected light. In diagnosis, the amount of in vivo components is important. However, physicians cannot accurately determine the amount of components based on images alone. For example, physicians estimate the amount of components to some extent by comparing the brightness of a target area where the amount of components is to be estimated with the brightness of other areas.

In photon-counting CT, a method of estimating in vivo components from the number of observed photons is proposed. With such a method, by using maximum likelihood estimation, the amount of in vivo components at each position is estimated from the number of photons based on a physical model. However, when there is an unknown component not taken into account in the physical model, the estimation may fail. A possible method to deal with such a case may be to use a physical model that takes unknown components into account. However, it increases the number of estimation targets and increases the likelihood of having a plurality of optimal solutions. As a result, “noise component caused by external factors” and “unknown component that is contained in a living body but not represented in a physical model” cannot be separated (distinguished), and such components may be represented as noise when imaged.

### BRIEF DESCRIPTION OF THE DRAWINGS

FIG. 1 is a block diagram illustrating an example of a configuration of a medical information processing system according to a first embodiment;

FIG. 2 is a diagram illustrating an example of a case where a plurality of components are depicted in a CT image, a component amount for each of the components is estimated, and reconstruction of the estimated component amounts is performed to generate a plurality of component maps;

FIG. 3 is a diagram illustrating an example of a case where a plurality of components are depicted in a CT image, a component amount for each of the components is estimated, and reconstruction of the estimated component amounts is performed to generate a plurality of component maps;

FIG. 4 is a diagram for describing an example of processing executed by a morphological feature evaluation function according to the first embodiment;

FIG. 5 is a diagram for describing an example of the processing executed by the morphological feature evaluation function according to the first embodiment;

FIG. 6 is a diagram for describing another example of the processing executed by the morphological feature evaluation function according to the first embodiment;

FIG. 7 is a diagram illustrating an example of an image for display according to the first embodiment;

FIG. 8 is a flowchart illustrating an example flow of processing executed by a medical information processing device according to the first embodiment;

FIG. 9 is a flowchart illustrating another example flow of the processing executed by the medical information processing device according to the first embodiment; and

FIG. 10 is a diagram illustrating an example of a configuration of an X-ray CT device according to a second embodiment.

### DETAILED DESCRIPTION

One of the issues that the embodiments disclosed in the specification and the drawings are to overcome is, in component estimation using a physical model that takes noise components and unknown components into account, to estimate “unknown component that is contained in a living body but not represented in a physical model” out of “noise component” and “unknown component that is contained in a living body but not represented in a physical model”. Note, however, that the issues to be solved by the embodiments disclosed in the specification and the drawings are not limited to the above issue. Issues related to the effects of the configurations indicated in the embodiments described below can also be positioned as other issues.

An X-ray CT device according to the embodiments includes an X-ray tube, an X-ray detector, a memory, and processing circuitry. The X-ray tube emits X-rays to a living body. The X-ray detector detects X-rays transmitted through the living body, and outputs detection data indicating a detection result. The processing circuitry: generates, from detection data, a medical image in which a living body is depicted; specifies a first address in a memory space of a memory; and writes the generated medical image to the specified first address; specifies the first address to read out the medical image written to the specified first address from the memory; estimates a component amount of an in vivo component and component amounts of a noise component and an unknown component based on the read-out medical image by using a physical model that takes a noise component and an unknown component into account; specifies a second address in the memory space of the memory; and writes the estimated component amount of the in vivo component as well as component amounts of the noise component and the unknown component to the specified second address; calculates likelihood of the component amounts with respect to the physical model as a first evaluation value; specifies a third address in the memory space of the memory; and writes the calculated first evaluation value to the specified third address; calculates a second evaluation value based on morphological features of the in vivo component, the noise component, and the unknown component; specifies a fourth address in the memory space of the memory; and writes the calculated second evaluation value to the specified fourth address; and specifies the second address to read out, from the memory, the component amount of the in vivo component and the component amounts of the noise component and the unknown component written to the specified second address; specifies the third address to read out the first evaluation value written to the specified third address from the memory; specifies the fourth address to read out the second evaluation value written to the specified fourth address from the memory; and updates the read-out component amount of the in vivo component as well as component amounts of the noise component and the unknown component based on the read-out first evaluation value and second evaluation value.

Hereinafter, the embodiments of the X-ray CT device, a medical image processing device, and a medical image processing method will be described in detail with reference to the accompanying drawings

### First Embodiment

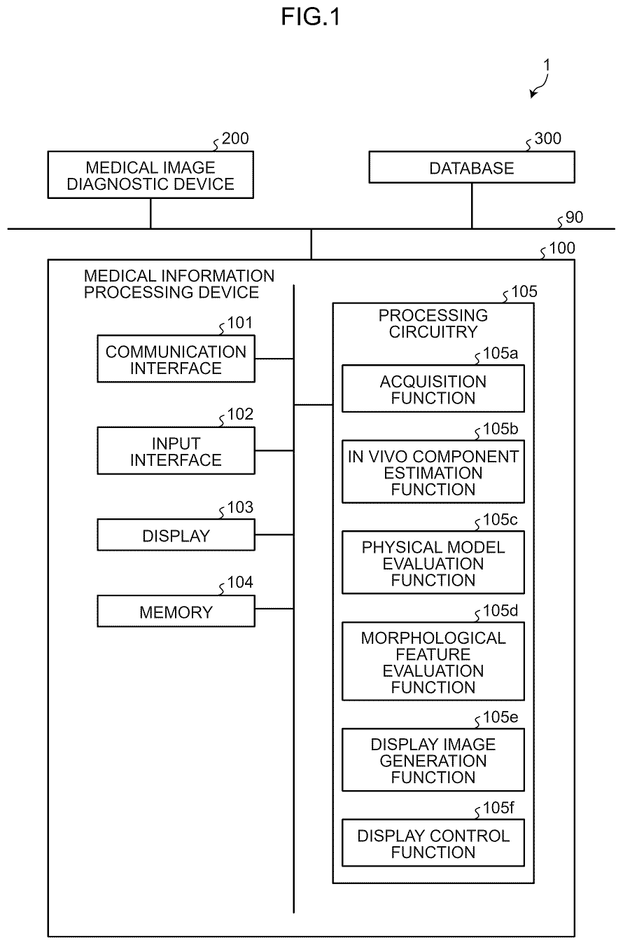

A first embodiment will be described by referring to a medical information processing system 1 including a medical information processing device 100 as an example. For example, the medical information processing system 1 includes the medical information processing device 100, a medical image diagnostic device 200, and a database 300, as illustrated in FIG. 1. FIG. 1 is a block diagram illustrating an example of the configuration of the medical information processing system 1 according to the first embodiment.

As illustrated in FIG. 1, the medical information processing device 100, the medical image diagnostic device 200, and the database 300 are connected via a network 90. Note here that the network 90 may be configured with a local network closed within a hospital, or may be a network via the Internet. For example, the network 90 includes a local area network (LAN) or a wide area network (WAN).

The medical image diagnostic device 200 is a device that collects medical images (medical image data) in which an inspection target site of a subject is depicted. That is, a medical image is an image in which a living body is depicted. The medical image diagnostic device 200 transmits medical images to the database 300 and the medical information processing device 100. The medical image diagnostic device 200 includes at least one of an X-ray CT device, an ultrasonic diagnostic device, a magnetic resonance imaging (MRI) device, and a visible light camera, for example. In a case where the medical image diagnostic device 200 is an X-ray CT device, the medical image diagnostic device 200 collects CT images (CT image data). Such an X-ray CT device is a device capable of executing photon counting CT, for example. In other words, such an X-ray CT device is a device that is capable of reconstructing X-ray CT images by counting photons of X-rays transmitted through the subject by using a photon-counting mode X-ray detector (photon-counting detector). In a case where the medical image diagnostic device 200 is an ultrasonic diagnostic device, the medical image diagnostic device 200 collects ultrasonic images (ultrasonic image data). In a case where the medical image diagnostic device 200 is an MRI device, the medical image diagnostic device 200 collects MR images (MR image data). In a case where the medical image diagnostic device 200 is a visible light camera, the medical image diagnostic device 200 collects optical images (optical image data). Hereinafter, a case in which the medical image diagnostic device 200 is an X-ray CT device capable of executing photon counting CT will be described as an example.

The database 300 is a storage device that stores therein various kinds of data, and it is implemented by a computer device such as a server or a workstation. The database 300 may be a server of an information management system such as a radiology information system (RIS), a hospital information system (HIS), or a picture archiving and communication system (PACS). For example, the database 300 includes an image storage device that stores therein medical images collected by the medical image diagnostic device 200. While a single database 300 is illustrated in FIG. 1, the database 300 may be implemented by a combination of a plurality of storage devices.

The medical information processing device 100 assists physicians in diagnosis. For example, the medical information processing device 100 performs component estimation using a physical model that takes unknown components into account. In component estimation using a physical model that takes unknown components into account, the medical information processing device 100 distinguishes between “noise” and “unknown component that is contained in a living body but not represented in a physical model”, mainly estimates “unknown component that is contained in a living body but not represented in a physical model”, and images and displays such a component. For example, the medical information processing device 100 is an example of a medical image processing device.

The medical information processing device 100 includes a communication interface 101, an input interface 102, a display 103, a memory 104, and processing circuitry 105, as illustrated in FIG. 1.

The communication interface 101 is configured with a network card such as a LAN card or a network adapter, for example. The communication interface 101 transmits and receives various kinds of information to and from devices connected via the network 90 under the control of the processing circuitry 105.

The input interface 102 receives various kinds of input operations from a user, converts the received input operations into electrical signals, and outputs those to the processing circuitry 105. For example, the input interface 102 can be implemented by a mouse, a keyboard, a trackball, switches, buttons, a joystick, a touchpad for making input operations by touching the operation surface, a touchscreen in which a display screen and a touchpad are integrated, a non-contact input circuit using an optical sensor, a voice input circuit, or the like. Note that the input interface 102 may be configured with a tablet terminal or the like capable of performing wireless communication with a main body of the medical information processing device 100. The input interface 102 may also be a circuit that receives input operations from the user through motion capture. Referring to an example, the input interface 102 can receive body movements, lines of sight, and the like of a user by processing signals acquired via the tracker and images collected about the user. The input interface 102 is not limited only to those with physical operation components such as a mouse and a keyboard. For example, electrical signal processing circuitry that receives electrical signals corresponding to input operations from an external input device provided separately from the medical information processing device 100, and outputs those electrical signals to the processing circuitry 105 is also an example of the input interface 102.

The display 103 displays various kinds of information and various kinds of images. For example, the display 103 displays various kinds of images such as medical images (medical images based on medical image data) collected by the medical image diagnostic device 200 under the control of the processing circuitry 105. Furthermore, the display 103 displays a graphical user interface (GUI) to receive various kinds of instructions, settings, and the like from the user via the input interface 102, for example. The display 103 is a liquid crystal display or a cathode ray tube (CRT) display, for example. Note that the display 103 may be a desktop type or may be configured as a tablet terminal or the like capable of performing wireless communication with the main body of the medical information processing device 100. The display 103 is an example of a display unit.

Note that the medical information processing device 100 may include a projector instead of or in addition to the display 103. The projector can project images onto a screen, a wall, a floor, and the like under the control of the processing circuitry 105. To give an example, the projector can also project images onto an arbitrary plane, object, space, or the like by projection mapping. Such a projector is an example of the display unit.

The memory 104 is implemented by a semiconductor memory element such as a random-access memory (RAM) or a flash memory, a hard disk, an optical disc, and the like. For example, the memory 104 stores therein various kinds of data (medical image data) such as various kinds of medical images transmitted from the medical image diagnostic device 200. Hereinafter, a case in which the memory 104 stores therein CT images transmitted from the medical image diagnostic device 200 will be described as an example. The CT images stored in the memory 104 are two-dimensional or three-dimensional image data. The memory 104 also stores therein computer programs for circuitry included in the medical information processing device 100 to implement various kinds of functions. The memory 104 may also be implemented by a server group (cloud) connected to the medical information processing device 100 via the network 90.

The processing circuitry 105 includes an acquisition function 105a, an in vivo component estimation function 105b, a physical model evaluation function 105c, a morphological feature evaluation function 105d, a display image generation function 105e, and a display control function 105f. The acquisition function 105a is an example of an acquisition unit. The in vivo component estimation function 105b is an example of an estimation unit as well as an example of an update unit. The physical model evaluation function 105c is an example of a first evaluation unit. The morphological feature evaluation function 105d is an example of a second evaluation unit. The display image generation function 105e is an example of a generation unit. The display control function 105f is an example of a display control unit.

In the medical information processing device 100 illustrated in FIG. 1, each processing function is stored in the memory 104 in the form of a computer program that can be executed by a computer. The processing circuitry 105 is a processor that reads out each computer program from the memory 104 and executes the computer program to implement the function corresponding to each computer program. In other words, the processing circuitry 105 after reading out each computer program comes to have each of the functions corresponding to the computer program that has been read out.

While it is described by referring to FIG. 1 that a single piece of processing circuitry 105 implements the acquisition function 105a, the in vivo component estimation function 105b, the physical model evaluation function 105c, the morphological feature evaluation function 105d, the display image generation function 105e, and the display control function 105f, the processing circuitry 105 may be configured with a combination of a plurality of independent processors and each processor may execute the computer program to implement the function. Furthermore, each of the processing functions of the processing circuitry 105 may be distributed or integrated into a single or a plurality of pieces of processing circuitry as appropriate.

The processing circuitry 105 may also use a processor of an external device connected via the network 90 to implement the functions. For example, the processing circuitry 105 reads out and executes computer programs corresponding to each of the functions from the memory 104 and uses the server group (cloud) connected to the medical information processing device 100 via the network 90 as computational resources to implement each of the functions illustrated in FIG. 1.

Note here that the processing circuitry 105 specifies an address in the memory space of the memory 104 when executing various kinds of processing, and writes various kinds of data, such as data used in various kinds of processing and data generated in various kinds of processing, to the specified address (the area of the memory 104 corresponding to the specified address). In addition, when executing various kinds of processing, the processing circuitry 105 specifies an address in the memory space of the memory 104 where data used for various kinds of processing is written, reads out the data written to the specified address from the memory 104, and executes various kinds of processing using the read-out data.

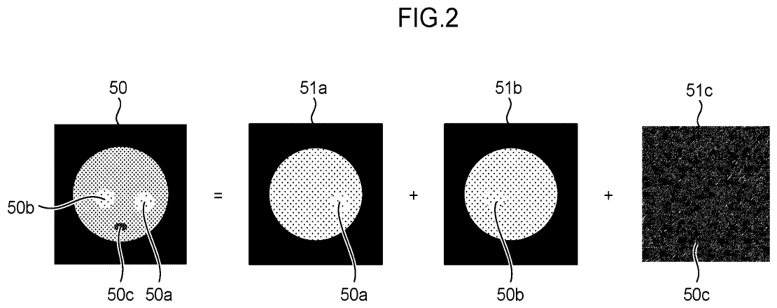

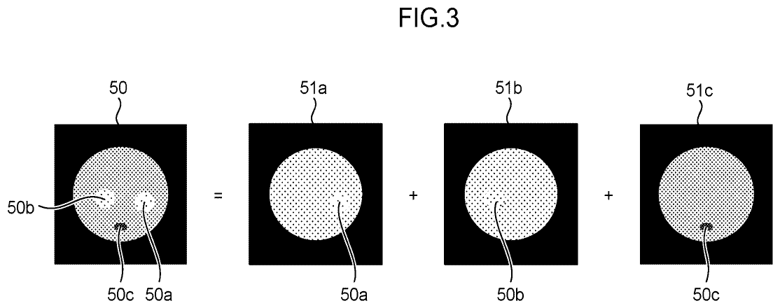

FIG. 2 and FIG. 3 are diagrams illustrating examples of a case where a component 50a, a component 50b, and a component 50c are depicted in a CT image 50, component amounts of each of the component 50a, the component 50b, and the component 50c are estimated, and reconstruction of the estimated component amounts is performed to generate three component maps 51a to 51c. Note, however, that the component 50c in FIG. 2 and the component 50c in FIG. 3 are different components. The component 50c in FIG. 2 is a noise component, whereas the component 50c in FIG. 3 is an unknown component that is contained in the living body (within the subject) but not represented in a physical model. The components 50a and 50b are components (components assumed in the physical model) that can be represented in the physical model used for estimating component amounts. The component 50c is a component considered in the physical model used for estimating the component amount.

As illustrated in FIG. 2 and FIG. 3, the component 50a and the component 50b, which are contained in the living body and represented in the physical model, have morphological features in the component map 51a and the component map 51b. However, as illustrated in FIG. 2, the component 50c, which is a noise component although it is a component considered in the physical model, becomes noise in the component map 51c and has no morphological features. On the other hand, as illustrated in FIG. 3, the component 50c, which is an unknown component contained in the living body and considered in the physical model, has morphological features in the component map 51c. In other words, the component 50c that is an unknown component exists in the living body, so that it has spatial features such as being locally distributed or distributed along blood vessels.

Therefore, based on the fact that the noise component and the unknown component contained in the living body have different features as described by referring to FIG. 2 and FIG. 3, in component estimation using a physical model that takes noise components and unknown components into account, the medical information processing device 100 according to the present embodiment distinguishes between “noise component” and “unknown component that is contained in the living body but not represented in the physical model”, mainly estimates and images “unknown component that is contained in the living body but not represented in the physical model” out of those components, as will be described later.

Next, examples of the processing executed by the acquisition function 105a, the in vivo component estimation function 105b, the physical model evaluation function 105c, the morphological feature evaluation function 105d, the display image generation function 105e, and the display control function 105f will be described.

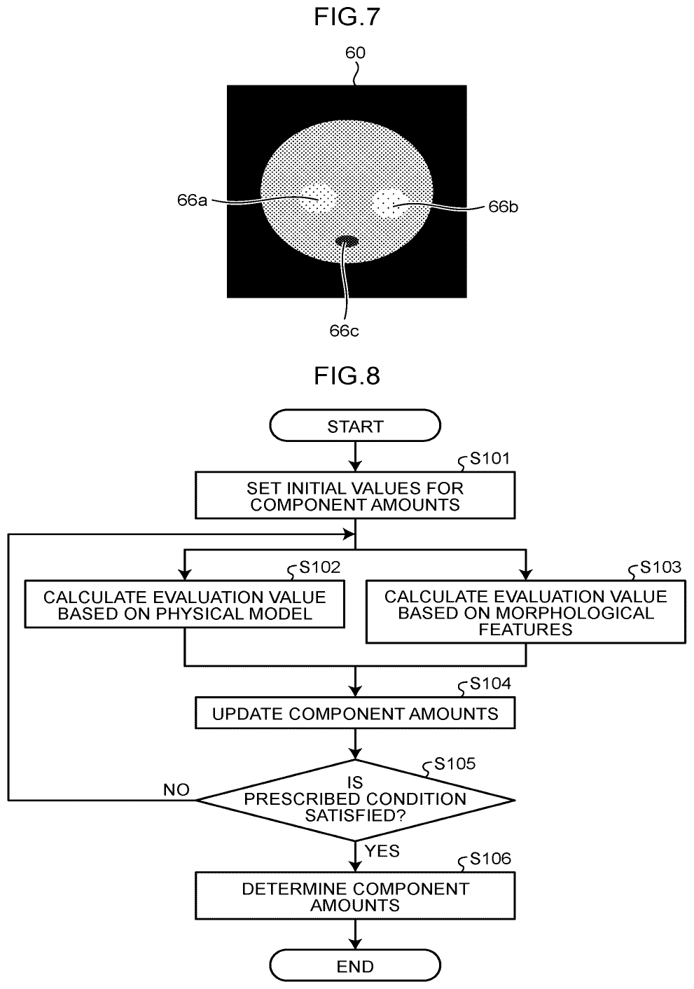

The acquisition function 105a acquires a CT image 60 (see FIG. 7) stored in the memory 104. The CT image 60 is used for various kinds of processing described later.

The in vivo component estimation function 105b estimates the component amounts in the living body using a physical model that takes noise components and unknown components into account. For example, the in vivo component estimation function 105b estimates the component amount at each position in the living body using a physical model indicated in the following Formula (1) (a physical model that takes noise components and unknown components into account).

$${λ}_{j}^{l} ( a ) = \int {w}_{j} ( E ) exp ( - {\sum }_{m = 1}^{M} {a}_{m} ( l ) {τ}_{m} ( E ) + ϵ ( l ) ) dE \qquad ( 1 )$$

In Formula (1), “l” is a value indicating the position of detection elements arranged in a detector provided in the X-ray CT device. Note that “j” is a value indicating a wavelength band (channel) of the X-rays. “E” is the energy of the X-rays. In addition, “λ^{l}ⱼ(a)” is an expected value of the number of photons of the X-rays in the wavelength band indicated by “j” incident on the detection element at the position indicated by “l”. Note that “wⱼ” is a weight (coefficient) corresponding to the X-rays in the wavelength band indicated by “j”. Also, “m” is a value indicating the kind of component. Furthermore, “aₘ” is a component amount of the kind of component indicated by “m”. Note that “τₘ” is a coefficient corresponding to the kind of component indicated by “m”. In addition, “ε” is a component amount of a noise component and an unknown component.

A method for estimating the component amount in a living body will be described in a specific manner. For example, “λ^{l}ⱼ(a)” is calculated in advance, and the in vivo component estimation function 105b uses “λ^{l}ⱼ(a)” to acquire an observed photon number yⱼ by applying a Poisson distribution to an expected value of the number of photons of the X-rays, λⱼ(a), according to the following Formula (2).

$${y}_{j} ∼ Poisson ( {λ}_{j} ( a ) ) \qquad ( 2 )$$

In Formula (2), “yⱼ” is the number of observed photons of the X-rays in the wavelength band indicated by “j”. Then, the in vivo component estimation function 105b estimates, as the component amount in the living body, the component amount aₘ when the value of an objective function Lₚ(a, ε) is minimized by minimizing the value of the objective function Lₚ(a, ε) indicated by the following Formula (3). Note that a is a vector of M-pieces of aₘ, that is, a=(a₁, a₂, . . . , a_{M}). In the present embodiment, the in vivo component estimation function 105b estimates the component amount aₘ by using the likelihood estimation/gradient descent method as the optimization method. However, the optimization method is not limited thereto. For example, the in vivo component estimation function 105b may estimate the component amount aₘ by using Bayesian optimization or a genetic algorithm.

$${L}_{p} ( a , ϵ ) = {\sum }_{j = 1}^{B} ( ( {λ}_{j} ( a , ϵ ) - {y}_{j} log ( {λ}_{j} ( a , ϵ ) ) ) \qquad ( 3 )$$

Note here that Formula (3) is a formula representing the objective function acquired from Formula (2). In Formula (3), Lₚ(a, ε) is the likelihood (degree of plausibility) of the component amount aₘ for the physical model indicated in Formula (1).

Then, the in vivo component estimation function 105b estimates a component amount ε of the noise component and the unknown component by calculating the component amount ε of the noise component and the unknown component from Formula (1) using “λ^{l}ⱼ(a, ε)” and the component amount aₘ.

Note that “to estimate each kind of component amount” is synonymous with “to break down a plurality of component amounts into each kind of component amount”. In the following description, an expression “break down a plurality of component amounts into each kind of component amount” may be used instead of an expression “estimate each kind of component amount”.

The physical model evaluation function 105c calculates an evaluation value based on the physical model indicated in Formula (1). For example, the physical model evaluation function 105c uses Formula (3) to calculate, as an evaluation value, the likelihood Lₚ(a, ε) of the component amount a with respect to the physical model indicated in Formula (1).

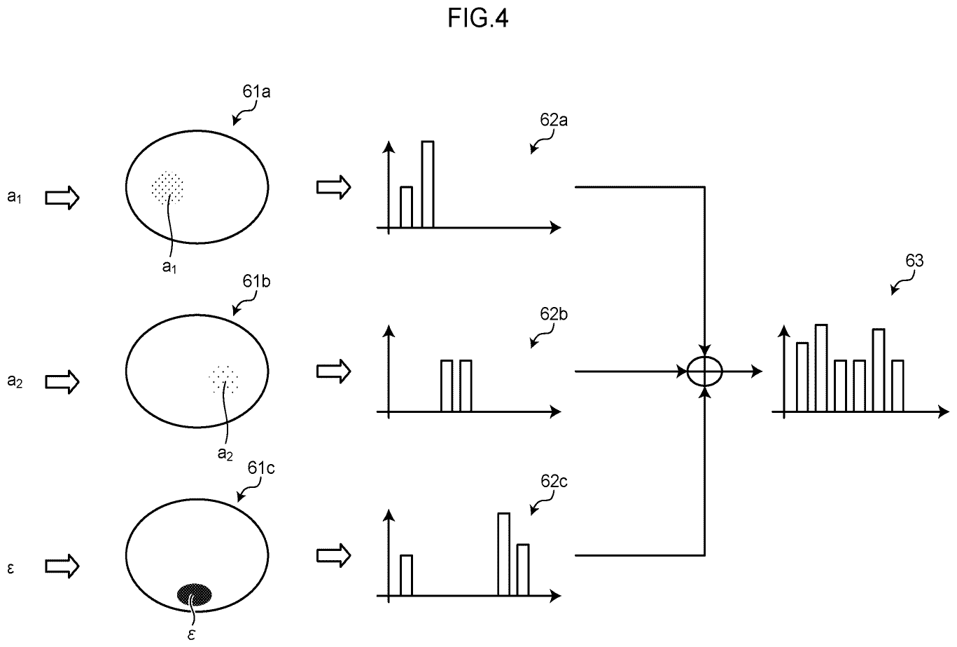

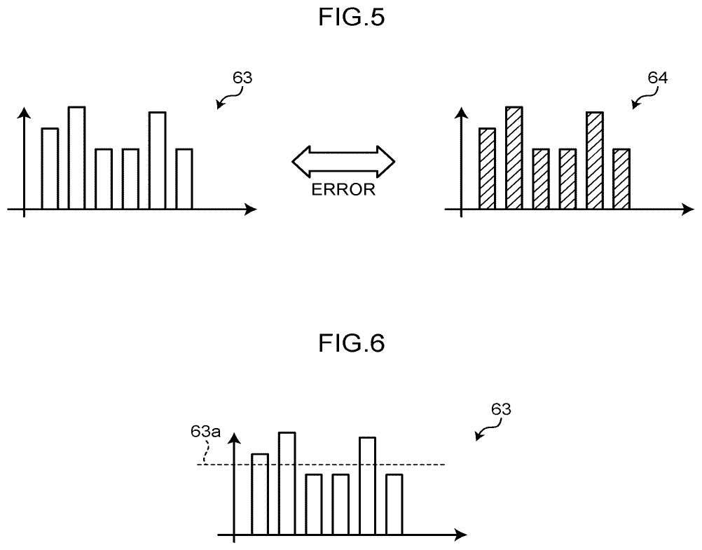

The morphological feature evaluation function 105d calculates an evaluation value based on morphological features. In the present embodiment, the morphological feature evaluation function 105d calculates an evaluation value regarding an in vivo component and evaluation values for a noise component and an unknown component. Hereinafter, a specific example will be described by referring to FIG. 4 and FIG. 5. FIG. 4 and FIG. 5 are diagrams for describing an example of the processing executed by the morphological feature evaluation function 105d according to the first embodiment. Here, a case in which a component amount a₁ and a component amount a₂ are estimated by the in vivo component estimation function 105b will be described as an example. Note that the component amount a₁ is the component amount of iodine, and the component amount a₂ is the component amount of bone. As illustrated in FIG. 4, the morphological feature evaluation function 105d performs reconstruction of the component amount a₁ to generate a component map 61a. Similarly, the morphological feature evaluation function 105d performs reconstruction of the component amount a₂ to generate a component map 61b, and performs reconstruction of the component amount ε to generate a component map 61c. In this manner, the morphological feature evaluation function 105d generates the component maps 61a to 61c by imaging the component amount a₁, the component amount a₂, and the component amount ε, respectively.

The morphological feature evaluation function 105d then extracts a morphological feature value for each of the component maps. As the morphological features, those that are less susceptible to noise and those that have additive characteristics are employed. For example, the morphological feature evaluation function 105d calculates higher-order local autocorrelation (HLAC) feature value or cubic higher-order local autocorrelation (CHLAC) feature value as morphological features from each of the component maps 61a to 61c. For example, the morphological feature evaluation function 105d calculates the HLAC feature value or the CHLAC feature value according to the following Formula (4).

$${x}^{N} ( {r}_{1} , {r}_{2} , … {r}_{N} ) = \int f ( s ) f ( s + {r}_{1} ) … f ( s + {r}_{N} ) ds \qquad ( 4 )$$

The number of kinds of elements (mask patterns) for correlation calculation is, for example, 35 kinds for the 0th-order, 1st-order, and 2nd-order features in a 3×3 pixel range. The HLAC feature value and the CHLAC feature value are less susceptible to noise since the autocorrelation of noise is zero. The HLAC feature value and the CHLAC feature value have additive characteristics of targets.

The morphological feature evaluation function 105d calculates the HLAC feature value or the CHLAC feature value for each element for each of the component maps 61a to 61c. Thereby, the morphological feature evaluation function 105d generates, for each of the component maps, a feature vector configured with the HLAC feature value or the CHLAC feature value for each element. That is, the morphological feature evaluation function 105d generates a feature vector 62a configured with the HLAC feature value or the CHLAC feature value for each element from the component map 61a. Similarly, the morphological feature evaluation function 105d generates a feature vector 62b from the component map 61b, and generates a feature vector 62c from the component map 61c.

The morphological feature evaluation function 105d then calculates the sum of all feature vectors 62a to 62c of all component maps 61a to 61c. In this manner, the morphological feature evaluation function 105d calculates a sum 63 of the feature vectors 62a to 62c by calculating the sum of the feature vector 62a, the feature vector 62b, and the feature vector 62c.

Then, as illustrated in FIG. 5, the morphological feature evaluation function 105d calculates a feature vector 64 by calculating the HLAC feature value or the CHLAC feature value for each element for the CT image (original image) 60. The morphological feature evaluation function 105d then compares the sum 63 of the feature vectors 62a to 62c with the feature vector 64, and defines an error Lₘ between the sum 63 of the feature vectors 62a to 62c and the feature vector 64 as the objective function using the following Formula (5).

$${L}_{m} ( a , ϵ ) = \frac{1}{D} {\sum }_{d = 1}^{D} ( {f}_{d} ( a , ϵ ) - \hat{{f}_{d}} ) \qquad ( 5 )$$

In Formula (5), “d” indicates an element, “D” indicates the number of all elements, “f_{d}” indicates the HLAC feature value or the CHLAC feature value of the element indicated by “d” in the sum 63 of the feature vectors 62a to 62c, “f^{λ}_{d}” indicates the HLAC feature value or the CHLAC feature value of the element indicated by “d” in the feature vector 64.

Note that the morphological feature evaluation function 105d may calculate the Euclidean distance (L2 norm) or L1 norm between the sum 63 of the feature vectors 62a to 62c and the feature vector 64 as the error. As the error, the morphological feature evaluation function 105d may also calculate the optimal transport distance that can take the shape of the distribution into account.

Here, in a case where the estimated component has morphological features in the component map, the error is relatively small. In a case where the estimated component does not have morphological features in the component map and appears as noise, the HLAC feature value or the CHLAC feature value becomes a value close to zero, so that the error becomes relatively large.

While the case in which the morphological feature evaluation function 105d calculates the evaluation value by comparing the sum 63 of the feature vectors 62a to 62c with the feature vector 64 is described, the morphological feature evaluation function 105d may calculate the evaluation value from the sum 63 of the feature vectors 62a to 62c without making such a comparison. An example of a method for calculating the evaluation value from the sum 63 of the feature vectors 62a to 62c will be described by referring to FIG. 6.

FIG. 6 is a diagram for describing another example of the processing executed by the morphological feature evaluation function 105d according to the first embodiment. For the element having the HLAC feature value or the CHLAC feature value greater than 0 in the sum 63 of the feature vectors 62a to 62c that are the morphological feature values, it is considered that the morphological features are reflected. This is because the HLAC feature value or the CHLAC feature value becomes a value close to zero for the noise. Thus, as illustrated in FIG. 6, the morphological feature evaluation function 105d may calculate, as the evaluation value, the number of elements for which the HLAC feature value or the CHLAC feature value becomes a threshold 63a or greater in the sum 63 of the feature vectors 62a to 62c.

The morphological feature evaluation function 105d may also calculate, as the evaluation value, the degree (likelihood) of the sum 63 of the feature vectors 62a to 62c fitting into a prescribed distribution (for example, mixed normal distribution).

The evaluation value Lₚ(a, ε) calculated by the physical model evaluation function 105c and the evaluation value Lₘ(a, ε) calculated by the morphological feature evaluation function 105d are used when updating the component amount a₁, the component amount a₂, and the component amount ε by the in vivo component estimation function 105b.

In the first embodiment, calculation of the evaluation value by the physical model evaluation function 105c, calculation of the evaluation value by the morphological feature evaluation function 105d, and update of the component amount a₁, the component amount a₂, and the component amount ε by the in vivo component estimation function 105b are repeatedly executed until a prescribed condition is satisfied. Then, when the prescribed condition is satisfied, the final component amount a₁, component amount a₂ and component amount ε are determined. The component amount ε determined in this manner is the component amount of the components in which the noise component is suppressed and the unknown component in the living body is the main component. Therefore, according to the first embodiment, it is possible to estimate “unknown component that is contained in a living body but not represented in a physical model” out of “noise component” and “unknown component that is contained in a living body but not represented in a physical model”.

The display image generation function 105e generates images for display. FIG. 7 is a diagram illustrating an example of an image for display according to the first embodiment; For example, the display image generation function 105e reconstructs each of the final component amount a₁, component amount a₂, and component amount ε to generate component maps 66a to 66c, respectively. Then, the display image generation function 105e applies different colors to each kind of components, which are colors with density corresponding to the component amounts for each of the component maps 66a to 66c. The display image generation function 105e then generates an image for display by superimposing each of the colored component maps 66a to 66c on the CT image 60. In FIG. 7, the component map 66c is an image based on the component amount ε of the components in which the noise component is suppressed and the unknown component in the living body is the main component. Therefore, according to the first embodiment, “unknown component that is contained in a living body but not represented in a physical model” can be mainly imaged out of “noise component” and “unknown component that is contained in a living body but not represented in a physical model”.

The display control function 105f displays various kinds of images on the display 103. For example, the display control function 105f displays the image for display illustrated in FIG. 7 (CT image 60 in which each of the colored component maps 66a to 66c is superimposed) on the display 103.

FIG. 8 is a flowchart illustrating an example flow of the processing executed by the medical information processing device 100 according to the first embodiment. As illustrated in FIG. 8, the in vivo component estimation function 105b sets randomly generated (determined) values or arbitrary values as initial values for the component amount aₘ in the living body and the component amount ε of the unknown component (step S101).

The physical model evaluation function 105c then calculates the evaluation value Lₚ(a, ε) based on the physical model indicated in Formula (1) (step S102). Furthermore, in parallel with the processing of step S102, the morphological feature evaluation function 105d calculates the evaluation value Lₘ(a, ε) based on morphological features (step S103).

Then, the in vivo component estimation function 105b calculates the evaluation value L(a, ε) by integrating the evaluation value Lₚ(a, ε) based on the physical model and the evaluation value Lₘ(a, ε) based on the morphological features and, based on the calculated evaluation value L(a, ε), updates the component amount aₘ in the living body and the component amount ε of the unknown component on the basis of optimization methods such as the gradient descent method and Bayesian optimization (step S104). For example, the in vivo component estimation function 105b updates the component amount aₘ in the living body and the component amount ε of the unknown component by adjusting the component amount aₘ in the living body and the component amount ε of the unknown component such that the evaluation value L(a, ε) becomes smaller. For example, with the gradient descent method, the gradient direction is calculated based on the derivative of the variables a and a in the evaluation value (evaluation function) L(a, ε), and the variables a and ε are updated based on the gradient direction such that L(a, ε) becomes smaller. Since the evaluation value L(a, ε) calculated by the total value of Formula (3) and Formula (5) is differentiable with respect to the variables a and ε, the in vivo component estimation function 105b can update the variables a and ε using the gradient descent method. Even when the evaluation value L(a, ε) is not differentiable with respect to the variables a and ε, the in vivo component estimation function 105b can make estimation using an optimization method such as Bayesian optimization. Note that the integrated evaluation value is calculated by a sum operation such as L(a, ε)=Lₚ(a, ε)+Lₘ(a, ε), for example. In a case where one is considered more important than the other, such as when the evaluation value based on the physical model is considered more important, weights wₚ and wₘ may be used to weight the values, as in L(a, ε)=wₚLₚ(a, ε)+ₘLₘ(a, ε).

The in vivo component estimation function 105b determines whether a prescribed condition is satisfied (step S105). For example, the prescribed condition is a condition that the processing of step S102, the processing of step S103, and the processing of step S104 are repeated a prescribed number of times. In this case, the in vivo component estimation function 105b determines whether the processing of step S102, the processing of step S103, and the processing of step S104 are repeated the predetermined number of times, and when determined that these pieces of processing are repeated the predetermined number of times (Yes at step S105), the in vivo component estimation function 105b shifts to step S106. On the other hand, when determined that these pieces of processing are not repeated the predetermined number of times (No at step S105), the in vivo component estimation function 105b shifts to step S102 and step S103. Then, returning to step S102 and step S103, the physical model evaluation function 105c calculates the evaluation value Lₚ(a, ε) based on the updated component amount aₘ and component amount ε at step S102. At step S103, the morphological feature evaluation function 105d calculates the evaluation value Lₘ(a, ε) based on the updated component amount aₘ and component amount ε.

Note that the prescribed condition may be a condition that the evaluation value L(a, ε) calculated at step S104 is equal to or smaller than a threshold. In this case, the in vivo component estimation function 105b determines whether the evaluation value L(a, ε) calculated at step S104 is equal to or smaller than the threshold, and when the evaluation value L(a, ε) is equal to or smaller than the threshold (Yes at step S104), the in vivo component estimation function 105b shifts to step S106. On the other hand, when the evaluation value L(a, ε) is greater than the threshold (No at step S104), the in vivo component estimation function 105b shifts to step S105.

As a premise, when a substance is depicted in a CT image, the CT image has some morphological features. Thus, it is desirable for an image representing each component (component image) to have morphological features. With component estimation in which morphological features are not taken into account, the component amounts are not updated to reflect the morphological features. As the update directions of the component amounts, there are both directions that are a direction not reflecting morphological features and a direction reflecting morphological features. When performing component estimation in which morphological features are not taken into account, a large amount of computation is required to generate component images that reflect morphological features since the update directions are not limited to the direction reflecting morphological features. For example, the processing of step S104 in FIG. 8 needs to be updated many times. In addition, when performing component estimation in which morphological features are not taken into account, morphological features may fail to be reflected since the update is performed without taking morphological features into account in the first place.

On the other hand, the medical information processing device 100 according to the first embodiment uses the evaluation value L(a, ε) for which the evaluation value Lₘ(a, ε) reflecting the morphological features is taken into account, so that the update directions of the component amounts are limited to the update direction reflecting the morphological features. Therefore, the medical information processing device 100 according to the first embodiment can estimate component images reflecting the morphological features with a small number of updates.

At step S106, the in vivo component estimation function 105b determines the final component amounts. For example, among the evaluation values L(a, ε) calculated at step S104, the in vivo component estimation function 105b determines the component amount aₘ and the component amount ε corresponding to the smallest evaluation value L(a, ε) as the final component amount aₘ and component amount ε. As an example, when the processing of step S102, the processing of step S103, and the processing of step S104 are repeated the prescribed number of times, a prescribed number of evaluation values L(a, ε) for the prescribed number of times are calculated at step S104. Therefore, the in vivo component estimation function 105b determines the component amount aₘ and the component amount ε corresponding to the smallest evaluation value L(a, ε) among the prescribed number of evaluation values L(a, ε) as the final component amount aₘ and component amount ε. The in vivo component estimation function 105b then ends the processing illustrated in FIG. 8.

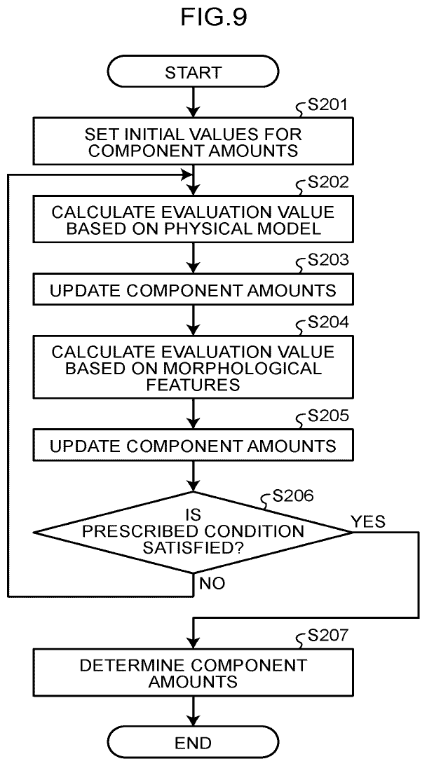

The processing illustrated in FIG. 8 is described regarding a case in which the processing of the physical model evaluation function 105c for calculating the evaluation value and the processing of the morphological feature evaluation function 105d for calculating the evaluation value are executed in parallel. However, the processing of the physical model evaluation function 105c for calculating the evaluation value and the processing of the morphological feature evaluation function 105d for calculating the evaluation value may also be executed at different timings. Thus, such processing will be described by referring to FIG. 9.

FIG. 9 is a flowchart illustrating another example flow of the processing executed by the medical information processing device 100 according to the first embodiment. As illustrated in FIG. 9, the in vivo component estimation function 105b sets randomly generated (determined) values or arbitrary values as initial values for the component amount am in the living body and the component amount ε of the unknown component (step S201).

The physical model evaluation function 105c then calculates the evaluation value Lₚ(a, ε) based on the physical model indicated in Formula (1) (step S202).

Then, the in vivo component estimation function 105b updates the component amount aₘ and the component amount ε based on the evaluation value Lₚ(a, ε) calculated at step S202 (step S203). At step S203, the in vivo component estimation function 105b updates the component amount aₘ and the component amount ε such that the evaluation value Lₚ(a, ε) becomes smaller by using the optimization methods such as the gradient descent method and Bayesian optimization.

Then, the morphological feature evaluation function 105d calculates the evaluation value Lₘ(a, ε) based on morphological features using the component amount aₘ and the component amount ε updated at step S203 (step S204).

Then, the in vivo component estimation function 105b updates the component amount aₘ and the component amount ε based on the evaluation value Lₘ(a, ε) calculated at step S204 (step S205). At step S205, the in vivo component estimation function 105b updates the component amount aₘ and the component amount ε such that the evaluated value Lₘ(a, ε) becomes smaller by using the optimization methods such as the gradient descent method and Bayesian optimization.

The in vivo component estimation function 105b then determines whether a prescribed condition is satisfied (step S206). For example, the prescribed condition is a condition that the processing of steps S202 to S205 is repeated a prescribed number of times. In this case, the in vivo component estimation function 105b determines whether the processing of steps S202 to S205 is repeated the predetermined number of times, and when determined that these pieces of processing are repeated the predetermined number of times (Yes at step S206), the in vivo component estimation function 105b shifts to step S207. On the other hand, when determined that these pieces of processing are not repeated the predetermined number of times (No at step S206), the physical model evaluation function 105c returns to step S202.

Note that the prescribed condition may be a condition that the evaluation value L(a, ε) calculated by integrating the evaluation value Lₚ(a, ε) calculated at step S202 and the evaluation value Lₘ(a, ε) calculated at step S204 is equal to or smaller than a threshold. In this case, at step S206, the in vivo component estimation function 105b calculates the evaluation value L(a, ε) by integrating the evaluation value Lₚ(a, ε) and the evaluation value Lₘ(a, ε), determines whether the calculated evaluation value L(a, ε) is equal to or smaller than the threshold, and when the estimation value L(a, ε) is equal to or smaller than the threshold (Yes at step S206), the in vivo component estimation function 105b shifts to step S207. On the other hand, when the evaluation value L(a, ε) is greater than the threshold (No at step S206), the physical model evaluation function 105c shifts to step S202. In addition, the prescribed condition may be a condition that the error calculated as the evaluation value at step S204 is equal to or smaller than a threshold. In this case, the in vivo component estimation function 105b determines whether the error calculated as the evaluation value at step S204 is equal to or smaller than the threshold and, when the error is equal to or smaller than the threshold (Yes at step S206), the in vivo component estimation function 105b shifts to step S207. On the other hand, when the error is greater than the threshold (No at step S206), the physical model evaluation function 105c returns to step S202.

Returning to step S202, the physical model evaluation function 105c calculates the evaluation value based on the physical model indicated in Formula (1) using the component amount aₘ and the component amount ε updated at step S205.

At step S207, the in vivo component estimation function 105b determines the final component amounts. For example, among the evaluation values L(a, ε) calculated at step S206, the in vivo component estimation function 105b determines the component amount aₘ and the component amount ε corresponding to the smallest evaluation value L(a, ε) as the final component amount aₘ and component amount ε. As an example, when the processing of steps S202 to S205 is repeated the prescribed number of times, the prescribed number of errors for the prescribed number of times are calculated as evaluation values at step S204. Therefore, the in vivo component estimation function 105b determines the component amount aₘ and the component amount ε corresponding to the smallest error among the prescribed number of errors as the final component amount aₘ and component amount ε. The in vivo component estimation function 105b then ends the processing illustrated in FIG. 9.

The medical information processing device 100 according to the first embodiment is described heretofore. The medical information processing device 100 includes: the acquisition function 105a for acquiring a medical image in which a living body is depicted; the in vivo component estimation function 105b for estimating the component amount of the in vivo component, component amounts of the noise component and unknown component using a physical model that takes noise components and unknown components into account; the physical model evaluation function 105c for calculating the likelihood of the component amount for the physical model as the first evaluation value; and the morphological feature evaluation function 105d for calculating the second evaluation value based on the morphological features of the in vivo component, the noise component, and the unknown component. Furthermore, the in vivo component estimation function 105b updates the component amount of the in vivo component and the component amounts of the noise component and the unknown component based on the first evaluation value and the second evaluation value. With the medical information processing device 100, as described above, it is possible to estimate “unknown component that is contained in a living body but not represented in a physical model” out of “noise component” and “unknown component that is contained in a living body but not represented in a physical model”.

In the example illustrated in FIG. 5, the morphological feature evaluation function 105d calculates the second evaluation value by comparing the morphological features of the in vivo component, the noise component, and the unknown component with the morphological features of the medical image data.

Furthermore, in the example illustrated in FIG. 6, the morphological feature evaluation function 105d calculates the second evaluation value based on the morphological features of the in vivo component, the noise component, and the unknown component.

Note that the morphological feature evaluation function 105d may calculate other feature values than the HLAC feature value and CHLAC feature value as long as they are feature values with the additive characteristics. For example, the morphological feature evaluation function 105d may calculate gradient local autocorrelation (GLAC) feature values or may calculate a simple histogram. The morphological feature evaluation function 105d may also divide the CT image 60 into local areas and extract features for each of the local areas by using the superpixel method.

The medical information processing device 100 may also execute the processing by switching the processing illustrated in FIG. 5 and the processing illustrated in FIG. 6. For example, the medical information processing device 100 updates the component amounts by executing the processing illustrated in FIG. 5 and, when the error calculated as the evaluation value is greater than the threshold even if the number of times for updating the component amounts is equal to or greater than a prescribed value, executes the processing illustrated in FIG. 6 to update the component amounts. In other words, the morphological feature evaluation function 105d switches the methods for calculating the second evaluation value in accordance with the condition.

### Second Embodiment

In the first embodiment, a case in which the medical information processing device 100 executes various kinds of processing is described. However, the X-ray CT device that is a medical image diagnostic device may perform the same processing as that executed by the medical information processing device 100 to estimate “unknown component that is contained in a living body but not represented in a physical model” out of “noise component” and “unknown component that is contained in a living body but not represented in a physical model”. Thus, such an embodiment will be described as a second embodiment. In the description of the second embodiment, the configurations that differ from those of the first embodiment will mainly be described and descriptions for the configurations similar to those of the first embodiment may be omitted.

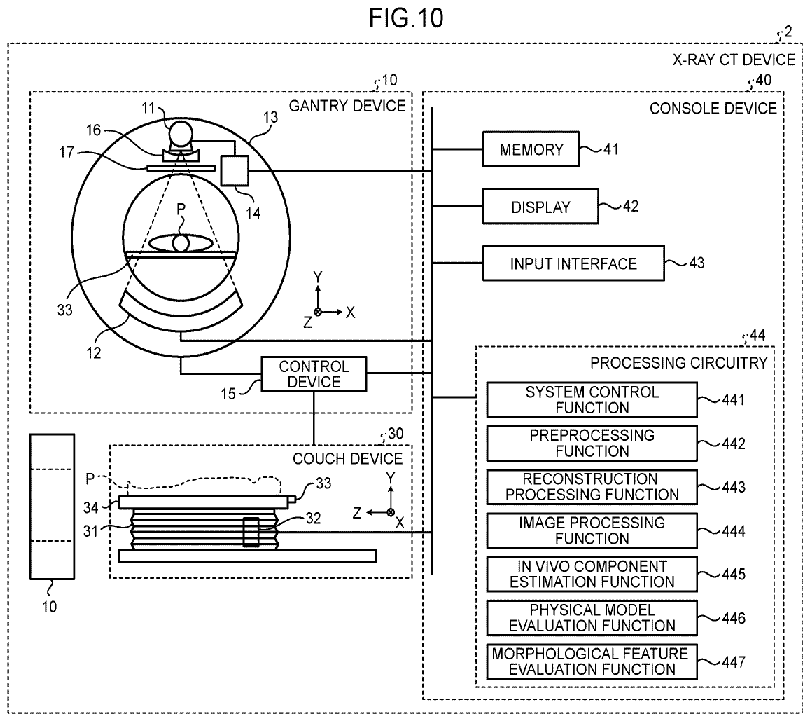

FIG. 10 is a diagram illustrating an example of the configuration of an X-ray CT device 2 according to the second embodiment. The X-ray CT device 2 is a device capable of executing photon counting CT, for example.

As illustrated in FIG. 10, the X-ray CT device 2 according to the present embodiment includes a gantry device 10, a couch device 30, and a console device 40, for example. For convenience of explanation, a plurality of gantry devices 10 are illustrated in FIG. 10.

In the present embodiment, the rotation axis of a rotation frame 13 or the longitudinal direction of a couchtop 33 of the couch device 30 in a non-tilted state is defined as a Z-axis direction, the axial direction that is orthogonal to the Z-axis direction and horizontal to the floor face is defined as an X-axis direction, and the axial direction that is orthogonal to the Z-axis direction and perpendicular to the floor face is defined as a Y-axis direction.

The gantry device 10 is a device that emits X-rays to a subject P (patient or the like), detects X-rays transmitted through the subject P, and outputs the X-rays to the console device 40. The subject P is an example of a living body. The gantry device 10 includes an X-ray tube 11, an X-ray detector 12, the rotation frame 13, a control device 15, a wedge 16, an X-ray aperture 17, and an X-ray high voltage device 14.

The X-ray tube 11 is a vacuum tube that generates X-rays by emitting thermoelectrons from a cathode (filament) toward an anode (target) through the application of high voltage from the X-ray high voltage device 14. For example, the X-ray tube 11 is a rotating anode X-ray tube that generates X-rays by emitting thermoelectrons onto a rotating anode.

The wedge 16 is a filter for adjusting the X-ray dose emitted from the X-ray tube 11. Specifically, the wedge 16 is a filter that transmits and attenuates the X-rays emitted from the X-ray tube 11 such that the X-rays emitted from the X-ray tube 11 to the subject P are applied in a distribution defined in advance. For example, the wedge 16 is a filter made of aluminum processed to be in a prescribed target angle and a prescribed thickness. The wedge 16 is also referred to as a wedge filter or a bow-tie filter.

The X-ray aperture 17 includes a lead plate or the like to narrow the irradiation range of the X-rays transmitted through the wedge 16, and a slit is formed by combining a plurality of lead plates or the like.

The X-ray detector 12 detects X-rays that are emitted from the X-ray tube 11 and transmitted through the subject P. Specifically, the X-ray detector 12 includes a plurality of detection elements that are arranged in the channel direction along a single arc centered at the focus of the X-ray tube 11. For example, the X-ray detector 12 has a structure in which a plurality of detection element rows, each row having a plurality of detection elements arranged in the channel direction, are arranged in a column direction (also referred to as a slice direction or a row direction). Note here that a collimator for reducing scattered X-rays is installed on the incident side of the X-rays of the X-ray detector 12. The collimator may also be referred to as an anti-scatter grid or a rear collimator.

Each of the detection elements outputs a signal that can measure the energy value of the photon of the X-rays each time the photon of the X-rays is incident. Specifically, the detection element is configured with a plurality of electrodes, and outputs an electrical signal corresponding to the incident X-rays each time the photon of the X-rays is incident. For example, zinc cadmium telluride (CZT: CdZnTe), cadmium telluride (CdTe), germanium (Ge), silicon (Si), and the like are used for the detection elements. Note that the detection element may be formed with other kinds of semiconductor crystals such as scintillator crystals.

In other words, the X-ray detector 12 is a direct conversion-type detector having a semiconductor element as the detection element that converts incident X-rays into electrical signals. Note that the X-ray detector 12 may be an indirect conversion-type detector that is a combination of a phosphor that is excited by X-rays and emits light, and an optical sensor that converts the light generated by the phosphor into an electrical signal.

The X-ray detector 12 includes signal processing circuitry that is connected to the detection elements described above and processes the electrical signals output from each of the detection elements. The signal processing circuitry counts the number of photons of the X-rays incident on the detection elements by performing pulse height discrimination of the pulses whose height is proportional to the individual electric charges of the electrical signals output from the detection elements. The signal processing circuitry also measures the energy of the counted photons of the X-rays by performing arithmetic processing based on the magnitude of the individual electric charges. Furthermore, the signal processing circuitry outputs the result of counting the number of photons of the X-rays as digital data signals by performing analog to digital (A/D) conversion of the signals from the detection elements. For example, the signal processing circuitry is implemented by an application specific integrated circuit (ASIC).

The X-ray detector 12 also includes a data acquisition system (DAS) that outputs detection data based on the signals output from the signal processing circuitry. The DAS generates detection data based on the signals of the result of counting the number of photons of the X-rays output from the X-ray detector 12. Note here that the detection data is data indicating the detection result. The detection data is also a sinogram, for example. A sinogram is the data in which the results of the processing that counts the number of photons incident onto each of the detection elements at each position of the X-ray tube 11 are arranged in order. Specifically, a sinogram is the data in which the results of counting the number of photons of the X-rays are arranged in a two-dimensional Cartesian coordinate system with the view direction and the channel direction being the axes. For example, the DAS generates a sinograms in a column unit in the slice direction of the x-ray detector 12. The DAS then transfers the generated detection data to the console device 40.

The X-ray high voltage device 14 includes: a high voltage generation device with electrical circuits such as a transformer and a rectifier, having a function of generating a high voltage to be applied to the X-ray tube 11; and an X-ray control device that controls the output voltage in accordance with the X-ray output emitted from the X-ray tube 11. The high voltage generation device may be a transformer type or an inverter type. The X-ray high voltage device 14 may be provided on the rotation frame 13 described below, or may be provided on a fixed frame (not illustrated) side of the gantry device 10. Note here that the fixed frame is a support frame that rotatably supports the rotation frame 13.

The rotation frame 13 is an annular frame that supports the X-ray tube 11 and the X-ray detector 12 in an opposing manner and rotates the X-ray tube 11 and the X-ray detector 12 by the control device 15 described later. Note that the rotation frame 13 further includes and supports the X-ray high voltage device 14, in addition to the X-ray tube 11 and the X-ray detector 12.

Note here that the rotation frame 13 is rotatably supported by the non-rotating part of the gantry device 10 (for example, the fixed frame that is not illustrated). The rotation mechanism includes, for example, a motor that generates a rotational drive force and a bearing that transmits the rotational drive force to the rotation frame 13 to rotate. The motor is provided at the non-rotating part, for example. The bearing is physically connected to the rotation frame 13 and the motor, and the rotation frame 13 is rotated in response to the rotational force of the motor.

Furthermore, the rotation frame 13 and the non-rotating part are each provided with a non-contact- or contact-type communication circuit, which enables communication between a unit supported by the rotation frame 13 and the non-rotating part or an external device of the gantry device 10. For example, when optical communication is employed as a non-contact communication method, the detection data generated by the DAS is transmitted from a transmitter having a light emitting diode (LED) provided in the rotation frame 13 to a receiver having a photodiode provided in the non-rotating part of the gantry device 10 by optical communication, and then further transferred by the transmitter from the non-rotating part to the console device 40. As the communication method, other non-contact data transmission methods such as capacitive coupling and radio wave methods, as well as a contact data transmission method using a slip ring and an electrode brush may also be used.

The control device 15 includes processing circuitry having a central processing unit (CPU) or the like, and a driving mechanism such as a motor and an actuator. The control device 15 has a function of performing operation control of the gantry device 10 and the couch device 30 by receiving input signals from an input interface 43 attached to the console device 40 or the gantry device 10. For example, the control device 15 performs the control for rotating the rotation frame 13, the control for tilting the gantry device 10, and the control for operating the couch device 30 and the couchtop 33 by receiving input signals. Note that the control for tilting the gantry device 10 by the control device 15 is achieved by rotating the rotation frame 13 about an axis parallel to the X-axis direction according to tilt angle information input via the input interface 43 attached to the gantry device 10. Note that the control device 15 may be provided in the gantry device 10 or may be provided in the console device 40.

The couch device 30 is a device for placing and moving the subject P that is a scan target, and it includes a base 31, a couch drive device 32, the couchtop 33, and a support frame 34. The base 31 is a casing that supports the support frame 34 to be movable in the vertical direction. The couch drive device 32 is a motor or an actuator that moves the couchtop 33 on which the subject P is placed in the direction of the major axis of the couchtop 33. The couchtop 33 provided on the top face of the support frame 34 is a plate on which the subject P is placed. In addition to the couchtop 33, the couch drive device 32 may also move the support frame 34 in the direction of the major axis of the couchtop 33.

The console device 40 is a device that receives operations of the X-ray CT device 2 from an operator and reconstructs CT image data using the detection data collected by the gantry device 10. The console device 40 includes a memory 41, a display 42, the input interface 43, and processing circuitry 44. While an example a case in which the console device 40 and the gantry device 10 are separate units is described herein, the gantry device 10 may include the console device 40 or some of the structural components of the console device 40.

The memory 41 is implemented by a semiconductor memory element such as a random-access memory (RAM) or a flash memory, a hard disk, an optical disc, and the like. The memory 41 stores therein projection data and CT image data, for example.

The display 42 displays various kinds of information. For example, the display 42 outputs medical images (CT images) generated by the processing circuitry 44, a graphical user interface (GUI) for receiving various kinds of operations from the operator, and the like. For example, the display 42 is a liquid crystal display or a cathode ray tube (CRT) display. The display 42 may be provided on the gantry device 10, for example. Furthermore, for example, the display 42 may be a desktop type or may be configured with a tablet terminal or the like capable of performing wireless communication with the main body of the console device 40. The display 42 is an example of the display unit.

The input interface 43 receives various kinds of input operations from the operator, converts the received input operations into electrical signals, and outputs those to the processing circuitry 44. For example, the input interface 43 receives a scan condition for collecting projection data, a reconstruction condition for reconstructing CT image data, an image processing condition for generating post-processed images from the CT images, and the like. For example, the input interface 43 can be implemented with a mouse, a keyboard, a trackball, switches, buttons, a joystick, and the like. Note that the input interface 43 may be provided on the gantry device 10, for example. Furthermore, for example, the input interface 43 may be configured with a tablet terminal or the like capable of performing wireless communication with the main body of the console device 40.

The processing circuitry 44 controls the operations of the entire X-ray CT device 2. The processing circuitry 44 specifies an address in the memory space of the memory 41 when executing various kinds of processing, and writes various kinds of data, such as data used in various kinds of processing and data generated in various kinds of processing, to the specified address (the area of the memory 41 corresponding to the specified address). In addition, when executing various kinds of processing, the processing circuitry 44 specifies an address in the memory space of the memory 41 where data used for various kinds of processing is written, reads out the data written to the specified address from the memory 41, and executes various kinds of processing using the read-out data.

For example, the processing circuitry 44 executes a system control function 441, a preprocessing function 442, a reconstruction processing function 443, an image processing function 444, an in vivo component estimation function 445, a physical model evaluation function 446, and a morphological feature evaluation function 447.

The system control function 441 controls various kinds of functions of the processing circuitry 44 based on input operations received from the operator via the input interface 43. For example, the system control function 441 controls CT scan executed in the X-ray CT device 2. The system control function 441 controls the generation and display of CT image data in the console device 40 by controlling the preprocessing function 442, the reconstruction processing function 443, the image processing function 444, the in vivo component estimation function 445, the physical model evaluation function 446, and the morphological feature evaluation function 447. Furthermore, the system control function 441 has the function similar to that of the display control function 105f. Note, however, that the system control function 441 displays various kinds of images and various kinds of information on the display 42 instead of the display 103. The system control function 441 is an example of a display control unit.

The preprocessing function 442 generates projection data by applying preprocessing such as logarithmic transformation processing, offset correction processing, sensitivity correction processing between channels, and beam hardening correction on the detection data output from the DAS of the X-ray detector 12. Note that the data before preprocessing (detection data) and the data after preprocessing may be collectively referred to as projection data.

The reconstruction processing function 443 generates CT images (reconstructed images) by performing reconstruction processing using filtered back projection, iterative reconstruction, or the like on the projection data generated by the preprocessing function 442. In this manner, the reconstruction processing function 443 collects CT images in which the inspection target site of the subject is depicted. The reconstruction processing function 443 is an example of a collection unit.

The image processing function 444 converts the CT image data generated by the reconstruction processing function 443 into tomographic image data of an arbitrary cross section or three-dimensional image data by known methods according to input operations received from the operator via the input interface 43. Note that generation of three-dimensional image data may be performed directly by the reconstruction processing function 443. The image processing function 444 also has the function similar to that of the display image generation function 105e. The image processing function 444 is an example of a generation unit.

The in vivo component estimation function 445 has the function similar to that of the in vivo component estimation function 105b. Furthermore, the physical model evaluation function 446 has the function similar to that of the physical model evaluation function 105c. The morphological feature evaluation function 447 also has the function similar to that of the morphological feature evaluation function 105d.

Note here that the processing circuitry 44 is implemented by a processor, for example. In this case, each of the processing functions of the processing circuitry 44 is stored in the memory 41 in the form of a computer program that can be executed by a computer. The processing circuitry 44 reads out each computer program from the memory 41 and executes the computer program to implement the function corresponding to each computer program. In other words, the processing circuitry 44 after reading out each computer program comes to have each of the functions indicated within the processing circuitry 44 illustrated in FIG. 1.

While it is described herein that a single piece of processing circuitry 44 implements each of the above-described processing functions, the processing circuitry 44 may be configured with a combination of a plurality of independent processors and each processor may execute the computer program to implement each processing function. Furthermore, each of the processing functions of the processing circuitry 44 may be distributed or integrated into a single or a plurality of pieces of processing circuitry as appropriate. Each processing function of the processing circuitry 44 may also be implemented by a mixture of a hardware such as a circuit and software. Furthermore, while an example of a case in which a single memory 41 stores therein the computer program corresponding to each of the processing functions is described herein, the embodiments are not limited thereto. For example, a plurality of storage circuits may be disposed in a distributed manner, and the processing circuitry 44 may read out and execute the corresponding computer programs from the individual storage circuits.

The second embodiment is described heretofore. Like the medical information processing device 100 according to the first embodiment, in component estimation using a physical model that takes noise components and unknown components into account, it is possible with the X-ray CT device 2 according to the second embodiment to estimate “unknown component that is contained in a living body but not represented in a physical model” out of “noise component” and “unknown component that is contained in a living body but not represented in a physical model”.

Furthermore, the computer program to be executed by the processor may be provided by being installed in advance in a read-only memory (ROM), a storage unit, or the like. Note that the computer program may be provided in a file of format that can be installed on the above-described devices or in an executable format by being recorded on a computer readable storage medium such as a compact disc (CD)-ROM, a flexible disk (FD), a CD-R (recordable), a digital versatile disc (DVD), or the like. Furthermore, the computer program may also be stored on a computer connected to a network such as the Internet, and provided or distributed by being downloaded via the network.

In addition, the structural components of each of the devices illustrated in the above-described embodiments are functional concepts and do not necessarily need to be physically configured as illustrated in the drawings. In other words, the specific form of distribution or integration of each of the devices is not limited to those illustrated in the drawings, and all or some of those can be functionally or physically distributed or integrated in an arbitrary unit in accordance with various kinds of load, use state, and the like. Furthermore, all or any of the processing functions performed by each of the devices can be implemented by the CPU and the computer program that is analyzed and executed by the CPU, or may be implemented by hardware using wired logic.

In component estimation using a physical model that takes noise components and unknown components into account, it is possible with at least one of the above-described embodiments to estimate “unknown component that is contained in a living body but not represented in a physical model” out of “noise component” and “unknown component that is contained in a living body but not represented in a physical model”.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An X-ray CT device comprising:
an X-ray tube configured to emit X-rays to a living body;
an X-ray detector configured to detect X-rays transmitted through the living body, and output detection data indicating a detection result;
a memory; and
processing circuitry configured to
generate, from the detection data, a medical image in which the living body is depicted, specify a first address in a memory space of the memory, and write the generated medical image to the specified first address,
specify the first address to read out the medical image written to the specified first address from the memory, estimate a component amount of an in vivo component and component amounts of a noise component and an unknown component based on the read-out medical image by using a physical model that takes a noise component and an unknown component into account, specify a second address in the memory space of the memory, and write the estimated component amount of the in vivo component as well as component amounts of the noise component and the unknown component to the specified second address,
calculate likelihood of the component amounts with respect to the physical model as a first evaluation value, specify a third address in the memory space of the memory, and write the calculated first evaluation value to the specified third address,
calculate a second evaluation value based on morphological features of the in vivo component, the noise component, and the unknown component, specify a fourth address in the memory space of the memory, and write the calculated second evaluation value to the specified fourth address, and
specify the second address to read out, from the memory, the component amount of the in vivo component and the component amounts of the noise component and the unknown component written to the specified second address, specify the third address to read out the first evaluation value written to the specified third address from the memory, specify the fourth address to read out the second evaluation value written to the specified fourth address from the memory, and update the read-out component amount of the in vivo component as well as component amounts of the noise component and the unknown component based on the read-out first evaluation value and second evaluation value.

2. The X-ray CT device according to claim 1, wherein the processing circuitry switches methods for calculating the second evaluation value in accordance with a condition.

3. The X-ray CT device according to claim 1, wherein the processing circuitry calculates the second evaluation value by comparing the morphological features of the in vivo component, the noise component, and the unknown component with a morphological feature of the medical image.

4. The X-ray CT device according to claim 1, wherein the processing circuitry calculates the second evaluation value based on the morphological features of the in vivo component, the noise component, and the unknown component.

5. A medical image processing device comprising:
a memory; and
processing circuitry configured to
acquire a medical image in which a living body is depicted, specify a first address in a memory space of the memory, and write the acquired medical image to the specified first address,
specify the first address to read out the medical image written to the specified first address from the memory, estimate a component amount of an in vivo component and component amounts of a noise component and an unknown component based on the read-out medical image by using a physical model that takes a noise component and an unknown component into account, specify a second address in the memory space of the memory, and write the estimated component amount of the in vivo component as well as component amounts of the noise component and the unknown component to the specified second address,
calculate likelihood of the component amounts with respect to the physical model as a first evaluation value, specify a third address in the memory space of the memory, and write the calculated first evaluation value to the specified third address,
calculate a second evaluation value based on morphological features of the in vivo component, the noise component, and the unknown component, specify a fourth address in the memory space of the memory, and write the calculated second evaluation value to the specified fourth address, and
specify the second address to read out, from the memory, the component amount of the in vivo component and the component amounts of the noise component and the unknown component written to the specified second address, specify the third address to read out the first evaluation value written to the specified third address from the memory, specify the fourth address to read out the second evaluation value written to the specified fourth address from the memory, and update the read-out component amount of the in vivo component as well as component amounts of the noise component and the unknown component based on the read-out first evaluation value and second evaluation value.

6. A medical image processing method comprising:
acquiring a medical image in which a living body is depicted, specifying a first address in a memory space of a memory, and writing the acquired medical image to the specified first address;
specifying the first address to read out the medical image written to the specified first address from the memory, estimating a component amount of an in vivo component and component amounts of a noise component and an unknown component based on the read-out medical image by using a physical model that takes a noise component and an unknown component into account, specifying a second address in the memory space of the memory, and writing the estimated component amount of the in vivo component as well as component amounts of the noise component and the unknown component to the specified second address;
calculating likelihood of the component amounts with respect to the physical model as a first evaluation value, specifying a third address in the memory space of the memory, and writing the calculated first evaluation value to the specified third address;
calculating a second evaluation value based on morphological features of the in vivo component, the noise component, and the unknown component, specifying a fourth address in the memory space of the memory, and writing the calculated second evaluation value to the specified fourth address; and
specifying the second address to read out, from the memory, the component amount of the in vivo component and the component amounts of the noise component and the unknown component written to the specified second address, specifying the third address to read out the first evaluation value written to the specified third address from the memory, specifying the fourth address to read out the second evaluation value written to the specified fourth address from the memory, and updating the read-out component amount of the in vivo component as well as component amounts of the noise component and the unknown component based on the read-out first evaluation value and second evaluation value.
